(19) United States
(12) Patent Application Publication
(10) Pub. No.: US 2003/0086916 A1
(43) Pub. Date: May 8, 2003

(54) Use of peroxynitrite scavengers or peroxynitrite formation inhibitors that do not diminish nitric oxide synthesis or activity to reverse or prevent premature vascular senescence

(76) Inventors: Michael S. Goligorsky, Mt. Vernon, NY (US); Jun Chen, Valhalla, NY (US)

Correspondence Address: JONES, TULLAR & COOPER, P.C., P.O. BOX 2266 EADS STATION, ARLINGTON, VA, 22202

(21) Appl. No.: 10/269,032

(22) Filed: Oct. 11, 2002

Related U.S. Application Data
(60) Provisional application No. 60/329,010, filed on Oct. 12, 2001.

(51) Int. Cl. A61K 38/44; A61K 31/555; A61K 31/445; A61K 31/416; A61K 31/353; A61K 31/198; A61K 31/192; A61K 35/78

(52) U.S. Cl. 424/94.4; 514/185; 514/410; 514/327; 514/407; 514/456; 514/561; 424/769; 514/569; 514/562

(57) ABSTRACT

Premature vascular senescence is reversed or prevented in tissue or cells by contacting the tissue or cells with a peroxynitrite scavenger or peroxynitrite formation inhibitor that does not diminish nitric oxide synthesis. This finds application in treatment of patients with a disorder associated with elevated levels of advanced glycation end products in blood or tissue, e.g., patients with end stage renal disease or poorly controlled diabetes, and in contacting vascular tissue or cells ex vivo to prevent occurrence of premature senescence.

### STATEMENT REGARDING FEDERALLY SPONSORED RESEARCH OR DEVELOPMENT

This invention was made at least in part with Government support under National Institutes of Health grants numbers DK45462 and DK54602. The Government has certain rights in the invention.

### CROSS-REFERENCE TO RELATED APPLICATION

This application claims the benefit, under 35 U.S.C. 119(e), of U.S. Provisional Application No. 60/329,010, filed on Oct. 12, 2001, the contents of which are incorporated herein by reference.

### BACKGROUND OF THE INVENTION

1. Field of the Invention

This application is directed to treating animals including humans with premature vascular senescence as manifested by elevated blood or tissue levels of advanced glycation end products, and in other embodiments, with preventing the occurrence of premature vascular senescence in vascular tissue or cells in vitro or ex vivo.

2. Description of Related Art

Previous to this invention, premature vascular senescence has not been associated with any specific pathological condition or considered a problem in tissue or cells ex vivo harvested for use for medical purposes.

PCT/US02/29850 filed Oct. 10, 2002 claims technology relating to premature vascular senescence. This PCT application relates to treating premature vascular senescence using hydroxyguanidines and pharmaceutically acceptable salts thereof.

### BRIEF SUMMARY OF THE INVENTION

The present invention is directed to a method of ameliorating premature endothelial cell senescence and/or vasculopathy with peroxynitrite scavengers and/or with peroxynitrite formation inhibitors that do not diminish nitric oxide synthesis or activity. The method comprises administering to endothelial cells an effective amount of a peroxynitrite scavenger such as an ebselen-class compound and/or a peroxynitrite formation inhibitor that does not diminish nitric oxide synthesis or activity such as a manganese chelate having superoxide dismutase activity. Treatment of cells may be in vitro, in vivo, or ex vivo.

The present invention finds use in tissue culture and engineering applications and in treatment of animals including humans. For example, endothelial cells may be grown on a matrix material such as collagen or collagen-coated stents with the aim of producing endothelial cell coated blood vessels or stents for use in vascular repair surgery. In order to allow for such blood vessels and stents to be developed and harvested, endothelial cells should be kept alive for maximum time periods and premature vascular senescence should be avoided. It is advantageous therefore to introduce a peroxynitrite scavenger or a peroxynitrite formation inhibitor that does not diminish nitric oxide synthesis or activity into such cell culture in order to forestall adverse effects of reactive oxygen species that tend to appear in cell cultures as a consequence of hypoxic and anoxic episodes. Premature senescence of vascular endothelial cells also occurs in vivo in response to the adverse effects of reactive oxygen species or the presence of advanced glycation products, and it is then advantageous to administer to the animal or patient a peroxynitrite scavenger or peroxynitrite formation inhibitor that does not diminish nitric oxide synthesis or activity.

In one embodiment, denoted the first embodiment, the invention herein provides a method of treating premature endothelial cell senescence and/or diabetic vasculopathy due to poorly controlled diabetes which method comprises administering to a patient in need of such treatment an effective amount of an ebselen-class compound.

In another embodiment, denoted the second embodiment, the invention herein provides a method of preventing or reversing the occurrence of premature senescence in vascular tissue or vascular cells comprising incubating the tissue or cells with a premature vascular senescence preventing effective amount of agent selected from the group consisting of ebselen-class compounds.

In another embodiment, denoted the third embodiment, the invention herein provides a method of treating premature endothelial cell senescence and/or diabetic vasculopathy which method comprises administering to a patient in need of such treatment an effective amount of agent selected from the group consisting of peroxynitrite formation inhibitors that do not diminish nitric oxide synthesis or activity.

In another embodiment, denoted the fourth embodiment, the invention herein provides a method of preventing or reversing the occurrence of premature senescence in vascular tissue or vascular cells comprising incubating the tissue or cells with a premature vascular senescence preventing effective amount of agent selected from the group consisting of peroxynitrite formation inhibitors that do not diminish nitric oxide synthesis or activity.

As used herein the terms “treat” or “treatment” are for purposes of and applied to inhibiting, preventing, or ameliorating disease and dysfunction.

As used herein, an ebselen-class compound may be functionally defined as any compound that scavenges peroxynitrite. Examples of ebselen-class compounds include but are not limited to sulfur-containing amino acids such as cystine, cysteine, or methionine, substituted with tellurium or selenium (e.g., in place of the sulfur of such compounds). Other examples include but are not limited to polyphenols and their derivatives including plant favonoids such as sinapic acid (i.e., 3,5-dimethoxy-4-hydroxycinnamic acid), quercetin, resorufin, and bark extracts containing hamamelitannin, phenolic acids such as caffeic, chlorogenic and ferulic acids, uric acid, 3-methyl-1-phenyl-2-pyrazolin-5-one (MCI-186), 5,10,15,20-tetrakis(2,4,6-trimethyl-3,5-disulphonatophenyl)-porphyrinato iron (III) (also called FeTMPS) and 5,10,15,20-tetrakis(N-methyl-4′-pyridyl)-porphyrinato iron (III) (also called FeTMPyP) and 2,3,6-tribromo-4,5-dihydroxybenz methyl ether (TDB, a product of a marine alga). As used herein, “ebselen-class compound” includes ebselen, which is 2-phenyl-1,2-benzisoselenazol-3(2H)-one.

As used herein, a peroxynitrite formation inhibitor that does not diminish nitric oxide synthesis or activity may be defined functionally as any compound that prevents or reduces peroxynitrite formation but does not substantially inhibit nitric oxide synthesis or scavenge nitric oxide. Examples of peroxynitrite formation inhibitors that do not diminish nitric oxide synthesis or activity include but are not limited to certain manganese metalloporphyrins such as [5,10,15,20-tetrakis(4-carboxyphenyl)-porphyrinato]manganese (III) chloride (i.e., MnTBAP) or manganese (III) mesotetrakis (N-ethylpyridinium-2-yl)porphyrin, Mn(II) complex with a bis(cyclohexylpyridine)-substituted macrocyclic ligand (referred to as M40403), salen-manganese complexes such as EUK-134, Cu,Zn-SOD that has been genetically engineered to include a positively charged glycine and arginine containing carboxy-terminal tail, hexamethylenediamine-congugated SOD, SOD entrapped in cationic liposomes, pegalated SOD, 4-hydroxytetramethyl-piperidine-1-oxyl (Tempol).

As used herein, the term “premature vascular senescence” is used to mean cell cycle arrest associated with the expression of senescence associated β-galactosidase and not associated with the attrition of telomeres and is characterized by the propensity of the said cells toward apoptotic death.

As used herein, the term “animals” includes mammals including humans.

### BRIEF DESCRIPTION OF THE DRAWING

The features and advantages of the present invention will become apparent from the following detailed description of a preferred embodiment thereof, taken in conjunction with the accompanying drawings, in which:

FIG. 1 shows the proportion of SA β-galactosidase-positive HUVECs cultured on glycated collagen (GC) and native collagen (NC) that was treated with ebselen, NOHA, MnTBAP, and L-arginine.

### DETAILED DESCRIPTION OF THE INVENTION

Turning now to the first embodiment of the invention, that is the embodiment of the invention directed at a method of treating an animal, e.g., a human patient, with a disorder characterized by premature vascular senescence and/or diabetic vasculopathy and/or associated with elevated levels of advanced glycation end products in blood or tissue, comprising administering to the animal a therapeutically effective amount of agent which is selected from the group consisting of premature vascular senescence ameliorating ebselen-class compounds.

Elevated levels of advanced glycation end products in blood are present when elevated levels of total plasma advanced glycation end products (AGE) and/or elevated levels of pentosidine and/or elevated levels of Amadori albumin and/or elevated levels of Amadori hemoglobin (Hgb A1c), are determined.

Normal blood level of AGE is equal or below 11.4±2.9 U/ml; elevated levels are considered to be any levels above 14.5 U/ml. Normal blood level of pentosidine is 1.63±0.07 pmol/mg protein or less with elevated levels considered to be any levels above 2 pmol/mg. Normal blood level of Amadori serum albumin is 20.9±4.0 U/ml; elevated levels are considered to be any levels above 39 U/ml. Normal level of Hgb A1c is 0.4% or less; elevated levels are considered to be any levels above 0.7%.

Total plasma AGE is determined as described in Chiavelli, F., et al, J. Pediatr. 34, 486491.

Pentosidine level is determined using HPLC techniques as described in Sugiyama, S., et al, J. Am. Soc. Nephrol. 9, 1681-1688 (1998).

Amadori serum albumin is determined by ELISA as described in Schalkwijk, C., et al, Diabetes 48, 2446-2453 (1999).

Amadori hemoglobin (Hgb A1c) is determined using routine clinical laboratory testing.

Elevated levels of advanced glycation end products in tissues are deemed to be present when positive immunohistochemical staining of the biopsy material at above normal levels can be demonstrated using antibodies against N-carboxymethyl-lysine (CML) or pentosidine, as detailed in N. Tanji et al, J. Am. Soc. Nephrol. 11: 1656-66, 2000.

Disorders associated with elevated blood or tissue levels of advanced glycation end products include chronic renal disease, poorly controlled diabetes, mellitus, end-stage renal disease, peripheral vascular disease, systemic lupus erythematosus, and Alzheimer's disease and other neurodegenerative diseases.

End-stage renal disease is characterized by creatinine clearance below 10 mg/dl, which is usually associated with severe anemia and patients are treated with, in most cases, hemodialysis or peritoneal dialysis. Poorly controlled diabetes mellitus, types 1 and 2, is characterized by abnormal glucose tolerance test, elevated fasting glucose levels (>120 mg/dl) and/or frank hyperglycemia. Active systemic lupus erythematosus is characterized by polyarthralgia, proteinuria (>200 mg/day), elevated blood pressure, and elevated titer of anti-DNA antibodies. Alzheimer's disease is characterized by the loss of cognitive functions in the absence of otherwise identifiable neurotoxic, structural or metabolic abnormalities. Chronic renal diseases are characterized by persistent proteinuria (>200 mg/day) and elevated blood pressure (>140/90 mm Hg). Peripheral vascular disease includes disorders affecting the arteries, veins and lymphatics of the extremities.

Turning now to the premature vascular senescence ameliorating ebselen-class compounds. Testing for whether an ebselen-class compound is a premature vascular senescence ameliorating ebselen-class compound is carried out as follows: Vascular endothelial cells are grown on a protein matrix containing advanced glycation end products, e.g., glucose-modified matrix proteins, e.g., Matrigel, in the presence or absence of the ebselen-class compound being tested. Signs of premature cell senescence are examined following a 3-5 day interval. The ebselen-class compound meets the test if premature cell senescence is ameliorated in the presence of the agent. Alternatively, vascular endothelial cells subjected to advanced glycation end products for a period of time to induce premature cell senescence are treated with the agent being tested in the continuous presence of advanced glycation end products. The ebselen-class compound meets the test if the treatment results in the reversal of premature cell senescence.

The premature vascular senescence ameliorating ebselen-class compounds are preferably premature vascular senescence ameliorating analogs of cystine, cysteine or methionine substituted with tellurium or selenium (e.g., in place of the sulfur of such compounds), ebselen itself, or polyphenols and their derivatives including plant favonoids such as sinapic acid (i.e., 3,5-dimethoxy-4-hydroxycinnamic acid), quercetin, resorufin, and bark extracts containing hamamelitannin, phenolic acids such as caffeic, chlorogenic and ferulic acids, uric acid, 3-methyl-1-phenyl-2-pyrazolin-5-one (MCI-186), 5,10,15,20-tetrakis(2,4,6-trimethyl-3,5-disulphonatophenyl)-porphyrinato iron (III) (also called FeTMPS) and 5,10,15,20-tetrakis(N-methyl-4′-pyridyl)-porphyrinato iron (III) (also called FeTMPyP) and 2,3,6-tribromo-4,5-dihydroxybenz methyl ether (TDB, a product of a marine alga).

As indicated above, the agents are administered in a therapeutically effective amount. This amount is a premature vascular senescence ameliorating amount, that is an amount reducing, reversing, or stopping the progression of premature vascular senescence. For treatment of end stage renal disease, the therapeutically effective amount is a premature vascular senescence ameliorating amount where premature vascular senescence amelioration is manifested by reduction in or stopping of the progression of symptoms of cardiovascular diseases, such as coronary artery disease, peripheral vascular disease, clotting and stenosis of arterio-venous fistula in patients with end stage renal disease on hemodialysis. For treatment of poorly controlled diabetes mellitus, chronic renal diseases, systemic lupus erythematosus and Alzheimer's disease and other neurodegenerative diseases, the therapeutically effective amount is a premature vascular senescence ameliorating amount where premature vascular senescence amelioration is manifested by reduction in or a stopping of the progression of symptoms of cardiovascular diseases, such as coronary artery disease or peripheral vascular disease, or in the case of Alzheimer's disease and neurodegenerative diseases, in stopping of the progression of symptoms of those diseases. For peripheral vascular disease, the therapeutically effective amount is a premature vascular senescence ameliorating amount where premature vascular senescence is manifested by a reduction in or a stopping of the progression of symptoms of peripheral vascular disease. Therapeutic amounts depend on the agent administered and can range, for example, from 0.01 μmol/kg to 2 mmol/kg. For peroxynitrite scavengers, administration can be, for example, of a loading dose, e.g., of 0.1-10 mg/kg, followed by 0.01 to 10 mg/kg/hr. Other suitable dosage information for peroxynitrite scavengers, including ebselen and ebselen-class compounds, is exemplified in the working examples hereinafter.

The ebselen-class compound can be administered in admixture with antioxidant agents and vitamins (e.g., ascorbate, alpha-tocopherol, vitamin B6, vitamin B12, folate (folic acid), carotenoids, coenzyme Q10, phytoestrogens (including isoflavonoids), butylated hydroxytoluene (BHT), butylated hydroxyanisole (BHA) and n-3 polyunsaturated fatty acids (PUFA)) with or without L-arginine or N^{ω}-hydroxy-L-arginine (or other hydroxyguanidine) supplementation (20 mg/kg every 4 hours), as a nutriceutical. Hydroxyguanidines and pharmaceutically acceptable salts thereof, as disclosed in PCT/US02/29850 filed Oct. 10, 2002, can also be administered with the ebselen-class compounds.

The routes of administration include oral, transdermal, intravenous, and intramuscular. Preferably, the ebselen-class compound is administered orally, the pharmaceutical compositions comprising an ebselen-class compound may also contain an inert diluent such as an assimilable edible carrier and the like, be in hard or soft shell gelatin capsules, be compressed into tablets, or may be an elixir, suspension, syrup or the like. Thus one or more ebselen-class compounds is compounded for convenient and effective administration in pharmaceutically effective amounts with suitable pharmaceutically acceptable carrier in a therapeutically effective dose.

When administered intravenously, the method comprises direct intravenous injection of an effective amount of an ebselen-class compound or addition of an effective amount of an ebselen-class compound to an established intravenous infusion solution. When administered intravenously to a patient, the ebselen-class compound may be combined with other ingredients, such as carriers, and/or dilutents and/or adjuvants. Typical carriers include a solvent or dispersion medium containing, for example, pH buffered isotonic aqueous solutions, ethanol, polyols such as glycerol, propylene glycol, polyethylene glycol, suitable mixtures thereof, surfactants or vegetable oils. Isotonic agents such as sugars or sodium chloride may be incorporated in pharmaceutical compositions for administration. There are no limitations on the nature of the other ingredients, except that they must be pharmaceutically acceptable, efficacious for their intended administration, and should not degrade the activity of the active ingredients of the compositions. Ebselen-class compounds may also be impregnated into transdermal patches or contained in subcutaneous inserts, preferably in liquid or semi-liquid form so that a therapeutically effective amount of such compound may be time-released into a subject.

The precise therapeutically effective amount (or dose) of ebselen or ebselen-class compound to be used in a method of treating a patient suffering from endothelial senescence and/or diabetic vasculopathy due to poorly controlled diabetes may be determined by the practioner based on the age, weight and/or gender of the subject, severity of the disease state, diet, time and frequency of administration, drug combination(s), and or sensitivities. As one means of determining an effective amount for a particular patient, the extent to which SA β-galactosidase histochemical staining is diminished in biopsy material can be used.

In vivo activity of peroxynitrite scavengers can be assayed on the basis of diminished nitrotyro sine content in patient's protein. Reference to a immunohistological assay for nitrotyrosine-modified protein assay may be conveniently made in J. S. Beckman et al. 1994 “Extensive nitration of protein tyrosines in human atherosclerosis detected by immunocytochemistry,” J. Biol. Chem. 375, 81-88. Since ebselen-class compounds may act transiently in vivo, re-administration of such compounds is preferred.

Peroxynitrite scavenging activity can also be determined using the method of Boveris in which decomposition of chemically synthesized peroxynitrite is followed by loss of chemiluminescent activity in standard reaction mixtures supplemented or not with peroxynitrite scavenger (Alvarez, S. et al. Ann. N.Y. Acad. Sci. 957, 271-273 (2002).

We turn now to the second embodiment herein, that is the embodiment directed at a method of preventing the occurrence of premature senescence in vascular tissue or vascular cells ex vivo comprising incubating the tissue or cells with a premature vascular senescence preventing effective amount of agent selected from the group consisting of premature vascular senescence preventing ebselen-class compounds.

The vascular tissue or vascular cells are preferably obtained from saphenous vein or mammary artery and are preferably endothelial cells.

The test for determining premature vascular senescence preventing ebselen-class compound is preferably carried out as follows: Vascular grafts are treated with ebselen-class compound being tested. Reduced stenotic and thrombotic complications as compared to untreated grafts indicates a premature vascular senescence preventing ebselen-class compound. The premature vascular senescence preventing agents are preferably the same as the premature vascular senescence ameliorating ebselen-class compounds of the first embodiment herein, i.e., premature vascular senescence ameliorating analogs of cystine, cysteine or methionine substituted with tellurium or selenium (e.g., tellurium or selenium in place of the sulfur of such compounds), ebselen itself, or polyphenols and their derivatives including plant favonoids such as sinapic acid (i.e., 3,5-dimethoxy-4-hydroxycinnamic acid), quercetin, resorufin, and bark extracts containing hamamelitannin, phenolic acids such as caffeic, chlorogenic and ferulic acids, uric acid, 3-methyl-1-phenyl-2-pyrazolin-5-one (MCI-186), 5,10,15,20-tetrakis(2,4,6-trimethyl-3,5-disulphonatophenyl)-porphyrinato iron (III) (also called FeTMPS) and 5,10,15,20-tetrakis(N-methyl-4′-pyridyl)-porphyrinato iron (III) (also called FeTMPyP) and 2,3,6-tribromo-4,5-dihydroxybenz methyl ether (TDB, a product of a marine alga).

The incubation is preferably carried out in a medium comprising saline or phosphate buffered saline at a temperature ranging from 4° C. to 37.5° C., preferably at 35° C., for a time period which is appropriate for the use to which the treated vascular tissue or cells are to be put, and is generally in the range of ½ hour to 4 weeks.

The premature vascular senescence preventing amount of premature vascular senescence preventing ebselen-class compound, that is the concentration of the ebselen-class compound to be provided in the incubation medium, is preferably determined by trying a plurality of increasing concentrations. Reduced stenotic and thrombotic complications as compared to untreated grafts indicates an appropriate concentration of premature vascular senescence preventing ebselen-class compound. The appropriate concentration will differ depending on what particular ebselen-class compound is used and typically ranges from 10 0.1 μM to 10 mM. For ebselen itself a preferred concentration in the incubation medium ranges from 10 μg/dl to 100 μg/dl.

When cells are treated in vitro (in vitro is intended to include, but is not limited to, cells in culture) or ex vivo, an effective amount of ebselen class compound may be determined by exposing cells to increasing levels of such compound, determining a level at which a reduction in peroxynitrite-mediated damage occurs, and correlating such level with administration of an effective amount of ebselen-class compound. A reduction in peroxynitrite-mediated damage may be evaluated by nitrotyrosine protein measurements. In addition, an observed reduction in the indicia of cell senescence may also be used as a means of determining an effective amount of ebselen-class compound to be administered to cells. For example, an effective amount of ebselen-class compound may be determined by exposing cells to increasing levels of such compound, determining a level at which there is an observed reduction in a marker for endothelial cell senescence, and correlating such a level with administration of an effective amount of ebselen-class compound. Examples of markers for endothelial cell senescence include senescence associated (SA) β-galactosidase and β-thymosin. Nitric oxide may be measured using an amperometric detection technique with NO-selective microelectrodes.

A particular use for the second embodiment is to provide cells for plating on a vascular stent to provide a non-thrombogenic surface. The cells may be attached to the stent by a biocompatible glue or other linking technology.

Other uses for the second embodiment include providing cultured cells on an artificial heart valve or for seeding on artificial vascular grafts for femoral-to-poplital bypass surgery in a patient with peripheral vascular disease, so that the patient experiences less thrombotic and atheroembolic complications.

An alternative to the stent treatment described above is to covalently bond the ebselen-class compound to a biodegradable polymer, e.g., polylactic acid, and to coat the product on the stent. The ebselen-class compounds may also be bound to any desired prosthetic devices and applied to xenografts and allografts.

We turn now to the third embodiment of the invention, that is the embodiment of the invention directed at a method of treating an animal, e.g., a human patient, with a disorder characterized by premature vascular senescence and/or diabetic vasculopathy or associated with elevated levels of advanced glycation end products in blood or tissue, comprising administering to the animal a therapeutically effective amount of agent which is selected from the group consisting of premature vascular senescence ameliorating peroxynitrite formation inhibitors that do not diminish nitric oxide synthesis or activity.

Disorders associated with premature vascular senescence and/or elevated blood or tissue levels of advanced glycation end products include chronic renal disease, poorly controlled diabetes mellitus, end-stage renal disease, peripheral vascular disease, systemic lupus erythematosus, and Alzheimer's disease and other neurodegenerative diseases.

Turning now to the premature vascular senescence ameliorating peroxynitrite formation inhibitors that do not diminish nitric oxide synthesis or activity. Testing for whether a peroxynitrite formation inhibitor that does not diminish nitric oxide synthesis or activity is a premature vascular senescence ameliorating compound is carried out as follows: Vascular endothelial cells are grown on a protein matrix containing advanced glycation end products, e.g., glucose-modified matrix proteins, e.g., Matrigel, in the presence or absence of the peroxynitrite formation inhibitor that does not diminish nitric oxide synthesis or activity being tested. Signs of premature cell senescence are examined following a 3-5 day interval. The peroxynitrite formation inhibitor that does not diminish nitric oxide synthesis or activity meets the test if premature cell senescence is ameliorated in the presence of the agent. Alternatively, vascular endothelial cells subjected to advanced glycation end products for a period of time to induce premature cell senescence are treated with the agent being tested in the continuous presence of advanced glycation end products. The peroxynitrite formation inhibitors that do not diminish nitric oxide synthesis or activity meet the test if the treatment results in the reversal of premature cell senescence.

The premature vascular senescence ameliorating peroxynitrite formation inhibitors that do not diminish nitric oxide synthesis or activity are preferably manganese metalloporphyrins such as [5,10,15,20-tetrakis(4-carboxyphenyl)-porphyrinato]manganese (III) chloride (i.e., MnTBAP) or manganese (III) mesotetrakis (N-ethylpyridinium-2-yl)porphyrin, Mn(II) complex with a bis(cyclohexylpyridine)-substituted macrocyclic ligand (referred to as M40403), salen-manganese complexes such as EUK-134, Cu,Zn-SOD that has been genetically engineered to include a positively charged glycine and arginine containing carboxy-terminal tail, hexamethylenediamine-congugated SOD, SOD entrapped in cationic liposomes, pegalated SOD, 4-hydroxytetramethyl-piperidine-1-oxyl (Tempol).

As indicated above, the agents are administered in a therapeutically effective amount. This amount is a premature vascular senescence ameliorating amount, that is an amount reducing, reversing, or stopping the progression of premature vascular senescence. For treatment of end stage renal disease, the therapeutically effective amount is a premature vascular senescence ameliorating amount where premature vascular senescence amelioration is manifested by reduction in or stopping of the progression of symptoms of cardiovascular diseases, such as coronary artery disease, peripheral vascular disease, clotting and stenosis of arterio-venous fistula in patients with end stage renal disease on hemodialysis. For treatment of poorly controlled diabetes mellitus, chronic renal diseases, systemic lupus erythematosus and Alzheimer's disease and other neurodegenerative diseases, the therapeutically effective amount is a premature vascular senescence ameliorating amount where premature vascular senescence amelioration is manifested by reduction in or a stopping of the progression of symptoms of cardiovascular diseases, such as coronary artery disease or peripheral vascular disease, or in the case of Alzheimer's disease and neurodegenerative diseases, in stopping of the progression of symptoms of those diseases. For peripheral vascular disease, the therapeutically effective amount is a premature vascular senescence ameliorating amount where premature vascular senescence is manifested by a reduction in or a stopping of the progression of symptoms of peripheral vascular disease. Therapeutic amounts depend on the agent administered and can range, for example, from 0.01 μmol/kg to 2 mmol/kg. For peroxynitrite formation inhibitors that do not diminish nitric oxide synthesis or activity, administration can be, for example, of a loading dose, e.g., of 0.1-10 mg/kg, followed by 0.01 to 10 mg/kg/hr. Other suitable dosage information for peroxynitrite formation inhibitors that do not diminish nitric oxide synthesis or activity are exemplified in the working examples hereinafter.

The peroxynitrite formation inhibitors that do not diminish nitric oxide synthesis or activity can be administered in admixture with antioxidant agents and vitamins (e.g., ascorbate, alpha-tocopherol, vitamin B6, vitamin B12, folate (folic acid), carotenoids, coenzyme Q10, phytoestrogens (including isoflavonoids), butylated hydroxytoluene (BHT), butylated hydroxyanisole (BHA) and n-3 polyunsaturated fatty acids (PUFA)) with or without L-arginine or N^{ω}-hydroxy-L-arginine (or other hydroxyguanidine) supplementation (20 mg/kg every 4 hours), as a nutriceutical. Hydroxyguanidines and pharmaceutically acceptable salts thereof, as disclosed in PCT/US02/29850 filed Oct. 10, 2002, can also be administered with the ebselen-class compounds. The peroxynitrite formation inhibitors that do not diminish nitric oxide synthesis or activity may also be combined with the ebselen-class compounds listed in the first embodiment to treat premature vascular senescence.

The routes of administration include oral, transdermal, intravenous, and intramuscular. Preferably, the peroxynitrite formation inhibitors that do not diminish nitric oxide synthesis or activity are administered orally. The pharmaceutical compositions comprising peroxynitrite formation inhibitors that do not diminish nitric oxide synthesis or activity may also contain an inert diluent such as an assimilable edible carrier and the like, be in hard or soft shell gelatin capsules, be compressed into tablets, or may be an elixir, suspension, syrup or the like. Thus one or more peroxynitrite formation inhibitors that do not diminish nitric oxide synthesis or activity is compounded for convenient and effective administration in pharmaceutically effective amounts with suitable pharmaceutically acceptable carrier in a therapeutically effective dose.

When administered intravenously, the method comprises direct intravenous injection of an effective amount of a peroxynitrite formation inhibitors that do not diminish nitric oxide synthesis or activity or addition of an effective amount of a peroxynitrite formation inhibitors that do not diminish nitric oxide synthesis or activity to an established intravenous infusion solution. When administered intravenously to a patient, the peroxynitrite formation inhibitors that do not diminish nitric oxide synthesis or activity may be combined with other ingredients, such as carriers, and/or dilutents and/or adjuvants. Typical carriers include a solvent or dispersion medium containing, for example, pH buffered isotonic aqueous solutions, ethanol, polyols such as glycerol, propylene glycol, polyethylene glycol, suitable mixtures thereof, surfactants or vegetable oils. Isotonic agents such as sugars or sodium chloride may be incorporated in pharmaceutical compositions for administration. There are no limitations on the nature of the other ingredients, except that they must be pharmaceutically acceptable, efficacious for their intended administration, and should not degrade the activity of the active ingredients of the compositions. Peroxynitrite formation inhibitors that do not diminish nitric oxide synthesis or activity may also be impregnated into transdermal patches or contained in subcutaneous inserts, preferably in liquid or semi-liquid form so that a therapeutically effective amount of such compound may be time-released into a subject.

The precise therapeutically effective amount (or dose) of peroxynitrite formation inhibitors that do not diminish nitric oxide synthesis or activity to be used in a method of treating a patient suffering from endothelial senescence and/or diabetic vasculopathy due to poorly controlled diabetes may be determined by the practioner based on the age, weight and/or gender of the subject, severity of the disease state, diet, time and frequency of administration, drug combination(s), and or sensitivities. As one means of determining an effective amount for a particular patient, the extent to which SA β-galactosidase histochemical staining is diminished in biopsy material can be used.

In vivo activity of peroxynitrite scavengers can be assayed on the basis of diminished nitrotyrosine content in patient's protein. Reference to a imunohistological assay for nitrotyrosine-modified protein assay may be conveniently made in J. S. Beckman et al. 1994 “Extensive nitration of protein tyrosines in human atherosclerosis detected by immunocytochemistry,” J. Biol. Chem. 375, 81-88. The level at which cells have a restoration of their ability to generate bioactive nitric oxide (NO) also correlates with administration of an effective amount of peroxynitrite formation inhibitors that do not diminish nitric oxide synthesis or activity. Since peroxynitrite formation inhibitors that do not diminish nitric oxide synthesis or activity may act transiently in vivo, re-administration of such compounds is preferred.

We turn now to the fourth embodiment herein, that is the embodiment directed at a method of preventing the occurrence of premature senescence in vascular tissue or vascular cells ex vivo comprising incubating the tissue or cells with a premature vascular senescence preventing effective amount of agent selected from the group consisting of premature vascular senescence preventing peroxynitrite formation inhibitors that do not diminish nitric oxide synthesis or activity.

The vascular tissue or vascular cells are preferably obtained from saphenous vein or mammary artery and are preferably endothelial cells.

The test for determining premature vascular senescence preventing peroxynitrite formation inhibitors that do not diminish nitric oxide synthesis or activity is preferably carried out as follows: Vascular grafts are treated with peroxynitrite formation inhibitors that do not diminish nitric oxide synthesis or activity being tested. Reduced stenotic and thrombotic complications as compared to untreated grafts indicates a premature vascular senescence preventing peroxynitrite formation inhibitors that do not diminish nitric oxide synthesis or activity.

The premature vascular senescence preventing agents are preferably the same as the premature vascular senescence ameliorating peroxynitrite formation inhibitors that do not diminish nitric oxide synthesis or activity of the third embodiment herein, i.e., premature vascular senescence ameliorating agents including but are not limited to manganese metalloporphyrins such as [5,10,15,20-tetrakis(4-carboxyphenyl)-porphyrinato]manganese (III) chloride (i.e., MnTBAP) or manganese (III) mesotetrakis (N-ethylpyridinium-2-yl)porphyrin, Mn(II) complex with a bis(cyclohexylpyridine)-substituted macrocyclic ligand (referred to as M40403), salen-manganese complexes such as EUK-134, Cu,Zn-SOD that has been genetically engineered to include a positively charged glycine and arginine containing carboxy-terminal tail, hexamethylenediamine-congugated SOD, SOD entrapped in cationic liposomes, pegalated SOD, 4-hydroxytetramethyl-piperidine-1-oxyl (Tempol).

The incubation is preferably carried out in a medium comprising saline or phosphate buffered saline at a temperature ranging from 4° C. to 37.5° C., preferably at 35° C., for a time period which is appropriate for the use to which the treated vascular tissue or cells are to be put, and is generally in the range of ½ hour to 4 weeks.

The premature vascular senescence preventing amount of premature vascular senescence preventing peroxynitrite formation inhibitors that do not diminish nitric oxide synthesis or activity, that is the concentration of the peroxynitrite formation inhibitors that do not diminish nitric oxide synthesis or activity to be provided in the incubation medium, is preferably determined by trying a plurality of increasing concentrations. Reduced stenotic and thrombotic complications as compared to untreated grafts indicates an appropriate concentration of premature vascular senescence preventing peroxynitrite formation inhibitors that do not diminish nitric oxide synthesis or activity. The appropriate concentration will differ depending on what particular peroxynitrite formation inhibitors that do not diminish nitric oxide synthesis or activity is used and typically ranges from 0.1 μM to 10 mM.

When cells are treated in vitro or ex vivo, an effective amount of peroxynitrite formation inhibitors that do not diminish nitric oxide synthesis or activity may be determined by exposing cells to increasing levels of such compound, determining a level at which a reduction in peroxynitrite mediated damage occurs, and correlating such level with administration of an effective amount of peroxynitrite formation inhibitors that do not diminish nitric oxide synthesis or activity. A reduction in peroxynitrite mediated damage may be evaluated by nitrotyrosine protein measurements. In addition, an observed reduction in the indicia of cell senescence may also be used as a means of determining an effective amount of peroxynitrite formation inhibitors that do not diminish nitric oxide synthesis or activity to be administered to cells. For example, an effective amount of peroxynitrite formation inhibitors that do not diminish nitric oxide synthesis or activity may be determined by exposing cells to increasing levels of such compound, determining a level at which there is an observed reduction in a marker for endothelial cell senescence, and correlating such a level with administration of an effective amount of peroxynitrite formation inhibitors that do not diminish nitric oxide synthesis or activity. Examples of markers for endothelial cell senescence include senescence associated (SA) galactosidase and β-thymosin. The level at which cells have a restoration of their ability to generate nitric oxide (NO) also correlates with administration of an effective amount of peroxynitrite formation inhibitors that do not diminish nitric oxide synthesis or activity. Nitric oxide may be measured using an amperometric detection technique with NO-selective microelectrodes.

A particular use for the fourth embodiment is to provide cells for plating on a vascular stent to provide a non-thrombogenic surface. The cells may be attached to the stent by a biocompatible glue or other linking technology.

Other uses for the fourth embodiment include providing cultured cells on an artificial heart valve or for seeding on artificial vascular grafts for femoral-to-poplital bypass surgery in a patient with peripheral vascular disease, so that the patient experiences less thrombotic and atheroembolic complications.

An alternative to the stent treatment described above is to covalently bond the peroxynitrite formation inhibitors that do not diminish nitric oxide synthesis or activity to a biodegradable polymer, e.g., polylactic acid, and to coat the product on the stent. The peroxynitrite formation inhibitors that do not diminish nitric oxide synthesis or activity may also be bound to any desired prosthetic devices and applied to xenografts and allografts.

Additionally, tissue or cells may be incubated with premature vascular senescence preventing effective amount of an agent selected from the group consisting of premature vascular senescence preventing ebselen-class compounds in addition to the peroxynitrite formation inhibitors that do not diminish nitric oxide synthesis or activity or with agents that are both ebselen-class compounds and peroxynitrite formation inhibitors that do not diminish nitric oxide synthesis or activity.

The invention herein is supported by the following background example and is illustrated by the following working examples.

### BACKGROUND EXAMPLE I

Detection of SA-β-galactosidase was carried out utilizing the histochemical staining method of Dimitri et al, Proc. Natl. Acad. Sci. USA 92, 9363-9367 as modified by Van der Loo, B., et al, Exp. Cell Res. 241, 309-315 (1998). SA β-galactosidase is a known senescence marker. En face SA β-galactosidase staining of aortas derived from age-matched Zucker diabetic and Zucker lean rats revealed that the former exhibited an uniform accumulation of senescent endothelial cells, especially at the branching points of daughter vessels—24 out of 24 branches studied showed SA β-galactosidase staining. This phenomenon occurred in 12 week and 21 week-old diabetic rats, but was undetectable in age-matched Zucker lean rats (0/24 branches examined).

### BACKGROUND EXAMPLE 2

Human umbilical vein endothelial cells (HUVEC) after four passages were plated on glycated collagen with or without the addition of 0.1 mM ebselen. Application was made on day 1, 2 hours after plating. The extent of SA β-galactosidase staining was evaluated on days 3 and 5 of culture on a glycated or native collagen matrix. In addition, to study the reversibility process, HUVEC were plated on glycated collagen for three days (time sufficient to induce premature senescence), and ebselen was added every 12 hours, starting on day 3, and the cells were examined on day 5. Glycated collagen resulted in a concentration-dependent increase in the proportion of SA β-galactosidase-positive cells after three days in culture. Addition of ebselen to the culture medium completely abolished the development of premature senescence in HUVEC grown on glycated collagen. Ebselen was able to reverse premature senescence at all dilutions of glycated collagen.

### EXAMPLE I

A forty-three year-old male with end stage renal disease due to glomerulonephritis (or systemic lupus erythematosus, or polycystic kidney disease, of focal segmental glomerulosclerosis, or amyloidosis, or rapidly progressive renal disease) on chronic hemodialysi's has a serum creatinine concentration of 10 mg/dl, hematocrit of 33%, blood pressure 175/105 mm Hg and shows one of the following signs of AGE accumulation: elevated level of pentosidine (2.5 pmol/mg) or elevated level of Amadori serum albumin (40 U/ml). (In some cases, renal biopsy will be performed, which will directly disclose the deposition of AGE in the renal parenchyma and increased proportion of SA-beta-galactosidase-stained endothelial cells). The patient has a history of coronary artery disease (CAD) with recent coronary artery bypass surgery; however, his graft shows signs of stenosis. In addition, the patient has his arteriovenous fistula revised 3 times due to clotting and stenosis. The patient is also complaining of a non-healing foot ulcer and intermittent claudication, both signs of peripheral vascular disease. The patient starts receiving ebselen 1-20 mg/kg thrice/day and 6 months later shows significant subjective improvement of coronary symptoms, renal disease, healing of foot ulcer, normalization of blood pressure and a decrease in pentosidine (1.7 pmol/mg) and/or Amodori serum albumin (30 U/ml). Similar results are obtained in similar patients who are administered other ebselen-class compounds.

### EXAMPLE II

A thirty year-old patient with type I (alternatively, a 60 year-old patient with type 2) diabetes mellitus, past medical history of myocardial infarction, peripheral vascular disease, hypertension, proteinuria and non-healing foot ulcer, is receiving insulin, but experiences poor therapeutic response. Patient's fasting blood glucose level is 200 mg/dl. Additional laboratory findings include elevated Hgb A1c level (8.5%) as well as one of the following signs of AGE accumulation: elevated level of pentosidine (2.3 pmol/mg) or elevated level of Amadori serum albumin (41 U/ml). (In some cases, renal biopsy will be performed, which will directly disclose the deposition of AGE in the renal parenchyma and increased proportion of SA-beta-galactosidase-stained endothelial cells). The patient is started on ebselen 1-20 mg/kg thrice/day and 6 months later shows significant subjective improvement of coronary symptoms (coronary angiogram may show either no further worsening of stenotic lesions or some degree of improvement), healing of foot ulcer, normalization of blood pressure and, a decrease in pentosidine (1.7 pmol/mg or less) and/or Amadori serum albumin (30 U/ml or less). Similar results are obtained in similar patients who are administered other ebselen-class compounds.

### EXAMPLE III

A sixty-eight year old man with no known medical conditions has been experiencing a sustained loss of short-term memory for the past three years. His CBC, electrolytes and other plasma metabolites are within normal range. There is no vitamin deficiency, no abnormalities in liver function tests, and no previous history of cerebro-vascular accidents. Head computerized tomographic study shows no evidence of brain atrophy. Based on these findings the patient is diagnosed with Alzheimer's disease. Therapy with ebselen or other ebselen-class compounds is initiated at doses of 1-20 mg/kg thrice/day alone or in combination with antioxidant agents and vitamins (e.g., ascorbate, alpha-tocopherol, vitamin B6, vitamin B12, folate (folic acid), carotenoids, coenzyme Q10, phytoestrogens (including isoflavonoids), butylated hydroxytoluene (BHT), butylated hydroxyanisole (BHA) and n-3 polyunsaturated fatty acids (PUFA)). Two years later the patient shows no deterioration and a partial improvement of symptoms related to the memory loss.

### EXAMPLE IV

Positive results similar to those obtained in Examples I, II and III are obtained when the same dosage of peroxynitrite formation inhibitors that do not diminish nitric oxide synthesis or activity is substituted for the ebselen or ebselen-class compound.

### EXAMPLE V

A sixty-five year-old woman with a defective heart valve is being prepared for a surgical replacement of the valve with a prosthetic device. A saphenous vein is obtained to harvest endothelial cells for expansion and seeding onto the surface of a artificial valve. Endothelial cells are isolated from the graft and cultured in a medium containing ebselen or ebselen-class compound at a concentration of 10-100 μg/ml at 35° C. for 2-3 weeks and cells are seeded onto the valve in the continuous presence of the same concentration of ebselen or ebselen-class compound. Thus prepared, the artificial valve is implanted. The patient experiences less thrombotic and atheroembolic complications and may require lower doses of anticoagulants than patients not receiving ebselen or ebselen-class compounds to maintain functioning of the artificial valve.

### EXAMPLE VI

A seventy-two year old man with peripheral vascular disease is undergoing an elective femoral-to-popliteal bypass surgery utilizing an artificial vascular graft. A saphenous vein is obtained to harvest endothelial cells for expansion and seed onto the surface of a graft. Endothelial cells are isolated from the graft and cultured in a medium containing ebselen or ebselen-class compound at a concentration of 10-100 μg/ml at 35° C. for 2-3 weeks, and cells are seeded onto the graft in the continuous presence of the same concentration of ebselen or ebselen-class compound. Thus prepared, the vascular graft is implanted. The patient experiences less thrombotic and atheroembolic complications and may require lower doses of anticoagulants than patients not receiving ebselen or ebselen-class compounds to maintain patency of the graft.

### EXAMPLE VII

Positive results similar to those obtained in Examples V and VI are obtained when the same dosage of peroxynitrite formation inhibitors that do not diminish nitric oxide synthesis or activity is substituted for the ebselen or ebselen-class compound.

### EXAMPLE VIII

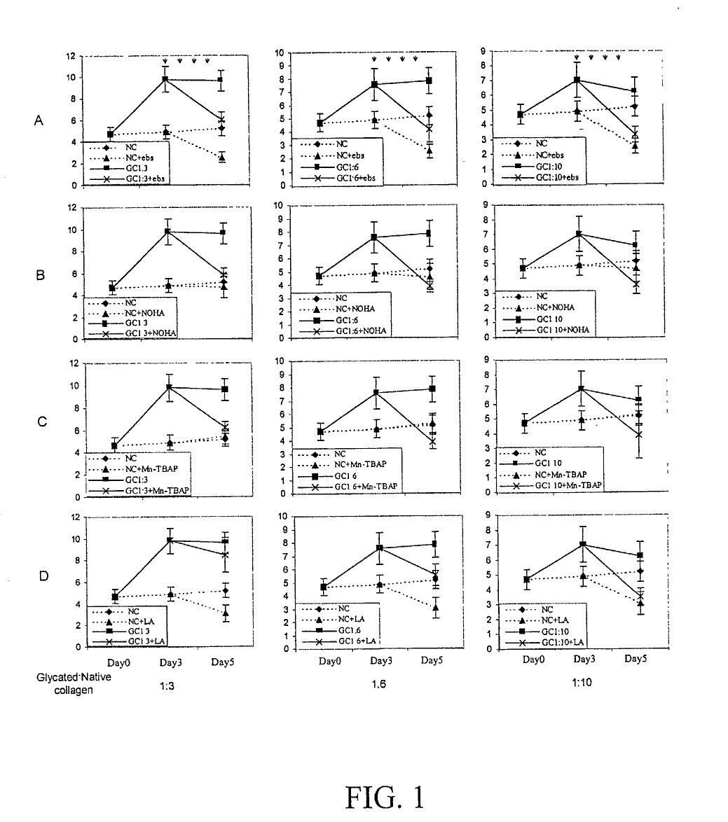

To elucidate the significance of enhanced peroxynitrite formation as an initiator of premature senescence of early-passage human umbilical vein endothelial cells (HUVECs), cells after 4 passages were plated on glycated collagen (GC) with and without the addition of the peroxynitrite scavenger, ebselen (ebs) (1 μmol/L), an intermediate in NO synthesis, 0.1 mmol/L N^{ω}-hydroxyl-L-arginine (NOHA) added to the same concentration as L-arginine, or a peroxynitrite formation inhibitor that does not diminish nitric oxide synthesis or activity (MnTBAP) (2.5 μmol/L). These concentrations of compounds were chosen based on preliminary experiments testing effects of a range of concentrations for each compound and selecting the lowest one which was efficient but non-cytotoxic. Ebselen, NOHA, and MnTBAP were each applied on day 1, 2 hours after HUVEC plating, added daily, and cells were studied on day 3. HUVECs grown on both glycated and native matrix were examined for the extent of SA β-galactosidase staining. Addition of ebselen, NOHA, or MnTBAP to the culture medium completely abolished the development of premature senescence in HUVECs grown on GC. In addition, to test for reversibility of senescence by ebselen, NOHA, or MnTBAP, HUVECs were plated on GC for 3 days (time sufficient to induce premature senescence) and thereafter ebselen (ebs), NOHA, or MnTBAP were added every 12 hours, until SA β-galactosidase staining on day 5. As shown in FIG. 1, growth on GC elicited a concentration-dependent increase in the proportion of SA β-galactosidase-positive cells by 3 days in culture. FIG. 1 shows treatments with ebselen (row A), NOHA (row B), MnTBAP (row C) and L-arginine (row D) and the dynamics of SA β-galactosidase-positive HUVECs cultured on different dilutions of GC (1:3, 1:6, and 1:10 respectively) mixed with native collagen (NC). Dashed lines show the data obtained in HUVECs cultured on native collagen (NC). Ebselen, NOHA, and MnTBAP were each able to reverse premature senescence at all dilutions of GC, in contrast to L-arginine (LA) alone, which reversed senescence at low but not high concentrations of GC.

Although the present invention has been disclosed in terms of a preferred embodiment, it will be understood that numerous additional modifications and variations could be made thereto without departing from the scope of the invention as defined by the following claims:

What is claimed is:

1. A method of treating an animal with premature vascular senescence comprising administering to the animal a therapeutically effective amount of an agent which is selected from the group consisting of premature vascular senescence ameliorating ebselen-class compounds.

2. The method of claim 1 where the animal has elevated levels of advanced glycation end products in blood or tissue

3. The method of claim 1 where the animal is affected with a disease selected from the group consisting of end stage renal disease, chronic renal disease and peripheral vascular disease.

4. The method of claim 1 where the animal is affected with poorly controlled diabetes.

5. The method of claim 1 where the animal is affected with systemic lupus erythematosis.

6. The method of claim 1 where the animal is affected with Alzheimer's disease or any other neurodegenerative disease.

7. The method of claim 1 where the animal is a human.

8. The method of claim 1, wherein the agent is selected from the group consisting of cystine, cysteine and methionine substituted with tellurium or selenium, polyphenols, flavonoids, plant polyphenols, sinapic acid, 3,5-dimethoxy-4-hydroxycinnamic acid, quercetin, resorufin, bark extracts containing hamamelitannin, phenolic acids, caffeic, chlorogenic and ferulic acids, uric acid, 3-methyl-1-phenyl-2-pyrazolin-5-one, 5,10,15,20-tetrakis(2,4,6-trimethyl-3,5-disulphonatophenyl)-porphyrinato iron (III), 5,10,15,20-tetrakis(N-methyl-4′-pyridyl)-porphyrinato iron (III) and 2,3,6-tribromo-4,5-dihydroxybenz methyl ether, TDB, and 2-phenyl-1,2-benzisoselenazol-3(2H)-one.

9. The method of claim 1, further comprising administering to the animal a therapeutically effective amount of an agent which is selected from the group consisting of premature vascular senescence ameliorating peroxynitrite formation inhibitors that do not diminish nitric oxide synthesis or activity.

10. A method for preventing the occurrence of premature senescence in vascular tissue or cells, comprising incubating the tissue or cells with a premature vascular senescence preventing effective amount of agent selected from the group consisting of premature vascular senescence preventing ebselen-class compound.

11. The method of claim 10, further comprising incubating the tissue or cells with a premature vascular senescence preventing effective amount of agent selected from the group consisting of premature vascular senescence ameliorating peroxynitrite formation inhibitors that do not diminish nitric oxide synthesis or activity.

12. The method of claim 10, further comprising treating said tissue or cells after seeding onto a substrate selected from the group consisting of a stent, an artificial heart valve, an artificial vascular graft, a xenograft, and an allograft.

13. A method of ameliorating senescence of vascular endothelial cells in vitro or ex vivo which comprises exposing said cells to an effective amount of an ebselen-class compound.

14. A method of treating an animal with premature vascular senescence comprising administering to the animal a therapeutically effective amount of an agent which is selected from the group consisting of premature vascular senescence ameliorating peroxynitrite formation inhibitors that do not diminish nitric oxide synthesis or activity.

15. The method of claim 14 where the animal has elevated levels of advanced glycation end products in blood or tissue

16. The method of claim 14 where the animal is affected with a disease selected from the group consisting of end stage renal disease, chronic renal disease, and peripheral vascular disease.

17. The method of claim 14 where the animal is affected with poorly controlled diabetes.

18. The method of claim 14 where the animal is affected with systemic lupus erythematosis.

19. The method of claim 14 where the animal is a human.

20. The method of claim 14 where the animal is affected with Alzheimer's disease or any other neurodegenerative disease.

21. The method of claim 14, wherein the agent is selected from the group consisting of manganese metalloporphyrins, [5,10,15,20-tetrakis(4-carboxyphenyl)-porphyrinato]manganese (III) chloride manganese (III) mesotetrakis (N-ethylpyridinium-2-yl)porphyrin, Mn(II) complex with a bis(cyclohexylpyridine)-substituted macrocyclic ligand, salen-manganese complexes, Cu,Zn-SOD that has been genetically engineered to include a positively charged glycine and arginine containing carboxy-terminal tail, hexamethylenediamine-congugated SOD, SOD entrapped in cationic liposomes, pegalated SOD, and 4-hydroxytetramethyl-piperidine-1-oxyl.

22. A method for preventing the occurrence of premature senescence in vascular tissue or cells, comprising incubating the tissue or cells with a premature vascular senescence preventing effective amount of agent selected from the group consisting of premature vascular senescence ameliorating peroxynitrite formation inhibitors that do not diminish nitric oxide synthesis or activity.

23. The method of claim 22, further comprising treating said tissue or cells after seeding onto a substrate selected from the group consisting of a stent, an artificial heart valve, an artificial vascular graft, a xenograft, and an allograft.

24. A method of ameliorating senescence of vascular endothelial cells in vitro or ex vivo which comprises exposing said cells to an effective amount of a peroxynitrite formation inhibitor that does not diminish nitric oxide synthesis or activity.
